(19) United States
(12) Patent Application Publication
(10) Pub. No.: US 2023/0260643 A1
(43) Pub. Date: Aug. 17, 2023

(54) SYSTEM AND PROCESS FOR MONITORING A PATIENT

(71) Applicant: Drägerwerk AG & Co. KGaA, Lübeck (DE)

(72) Inventors: Götz KULLIK, Lübeck (DE); Hinrich ALTHOFF, Lübeck (DE); Birger LANDWEHR, Lübeck (DE)

(21) Appl. No.: 18/166,039

(22) Filed: Feb. 8, 2023

(30) Foreign Application Priority Data
Feb. 11, 2022 (DE) 10 2022 103 208.6

(51) Int. Cl. G16H 40/63; G16H 10/60; G16H 50/30

(52) U.S. Cl. CPC G16H 40/63; G16H 10/60; G16H 50/30

(57) ABSTRACT

A system and process monitor a patient with an evaluation unit (1), a first interface (2) for receiving patient status values from a plurality of medical devices (3) to be available to the evaluation unit (1). The evaluation unit (1) generates a signal with patient-specific information based on the received patient status values and a further setpoint and/or actual value and transmits the signal to a receiver unit (5) via the first and/or a second interface (4). The evaluation unit (1) includes a suggestion module (6) that determines a suggested value which corresponds to a limit value for at least one patient status value based on the patient status values obtained from at least two medical devices (3) and the further setpoint and/or actual value, and to transmit this suggested value to the receiving unit (5) via the first and/or the at least one second interface (2, 4).

### CROSS REFERENCE TO RELATED APPLICATIONS

This application claims the benefit of priority under 35 U.S.C. § 119 of German Application 10 2022 103 208.6, filed Feb. 11, 2022, the entire contents of which are incorporated herein by reference.

### TECHNICAL FIELD

The invention relates to a system and a process for monitoring a patient. The system has at least one evaluation unit with a first interface, via which patient status values from at least two medical devices can be transmitted in parallel or serially to the evaluation unit. The evaluation unit is also configured to generate a signal with patient-specific information, taking into account the patient status values obtained and at least one further setpoint or actual value, and to transmit this signal to a receiver unit via the first and/or at least one second interface.

### BACKGROUND

When monitoring a patient, a large number of vital parameters or patient status values are regularly recorded and used in an evaluation unit of at least one medical device to determine the patient's condition. The available data is often provided by different devices, for example a heart-lung machine, a ventilator and a syringe pump, which in particular records information about the amount of medication supplied to a patient, for the purpose of monitoring and adjusting a patient's therapy as required. Intensive care units in particular have various therapy, monitoring and analysis devices, such as ventilators, patient monitors, syringe pumps, devices for blood gas analysis, food pumps and medical devices for diagnostic imaging.

With regard to future technical developments, a trend towards greater networking of the various medical devices provided in an intensive care unit can also be expected, with major advantages particularly in terms of an effective alerting of treatment staff.

In general, it is conceivable in this context that alarms generated by the medical devices used in monitoring a patient are verified based on the data provided by different medical devices and thus, for example, the alarm of one device is suppressed as soon as the data from a second medical device can be used to infer a false alarm.

However, in addition to the advantages offered by particularly comprehensive monitoring of a patient with a large number of different medical devices, both the amount of information provided by these devices and the noise generated pose a not inconsiderable problem in intensive care units. For example, as the number of devices used increases, so does the noise pollution for patients and treatment staff, both because of the greater number of technical components operated, such as fans and drive motors, and because of the large number of alarms and other acoustic signal tones. Furthermore, there is a risk that the large number of alarms may lead to a certain degree of alarm fatigue on the part of the treatment personnel, so that important alarms may not be correctly differentiated from supposedly less important alarms, resulting in an increase in the risk of danger to a monitored patient.

With regard to the aforementioned problem, it is of considerable importance to limit the total number of alarms issued to the required level and to set the limit values for the various medical devices, above which an alarm is triggered, to a reliable value. In this way, on the one hand, a risk to the patient should be excluded or at least further minimized and, on the other hand, the treatment staff should be prevented from being overburdened by an excessive number of alarms.

In this context, a system for changing alarm limits depending on the implementation of a certain treatment or therapy is known from U.S. Pat. No. 9,607,495 B2. For this purpose, a change to an alarm limit or an interval between permissible maximum and minimum limit values is proposed, for example, as a function of a treatment or a medication.

Furthermore, U.S. Pat. No. 10,537,290 B2 describes a system in which vital data, alarm limit changes and signaled alarms are evaluated in an evaluation unit from logbooks of several patient monitors and alarm limits which appear to be reasonable are determined on the basis of this evaluation. These alarm limits, which appear to make sense, are made available as suggested values to a central monitoring unit and can then be confirmed by the treatment staff and thus adopted by a medical device used for the treatment and/or monitoring of a patient.

In addition, a process and a system for providing needs-based alarm limits for patient monitoring are known from US 2019/295696 A1. With the aid of at least one sensor, vital parameters of a patient are recorded and fed to an evaluation unit. A so-called alarm setting classifier is provided in the evaluation unit, with which the current vital parameters of the monitored patient are evaluated and suggestions for alarm limits that appear suitable are made to the treatment personnel via a receiver unit.

Overall, the systems known from the prior art for monitoring a patient still have a disadvantage in that a large number of vital parameters or patient status values are often recorded by various medical devices and made available for evaluation, and attempts are also made to use the recorded patient status values for optimizing the alarm, but different monitoring systems are regularly only operated side by side. Since, in particular, no suitable exchange of data between the individual medical devices is provided, this means for the treatment staff that a multitude of information supplied by different devices and in many cases specific to the respective device and the therapy it performs must always be taken into account when assessing the condition of a patient and, above all, when setting alarm limits. Although in many cases the known systems record a number of vital signs and in some cases make suggestions to the treatment staff for reasonable alarm limits, different monitoring systems, which operate largely autonomously, must always be kept in view and taken into account when alarm limits are set. As a result, although a great deal of information is available for patient monitoring, it is not used effectively and therefore a holistic view of a patient's state of health can only be taken at comparatively great expense.

### SUMMARY

Based on the solutions known from the prior art for at least partially automated monitoring of a patient, in particular a patient in an intensive care unit, and the problems described above, the invention is based on an object of specifying a system and a process for patient monitoring in which the measured values and other data supplied by a large number of medical devices, in particular treatment, diagnostic and monitoring devices, can be used in an effective manner for assessing the state of health of a patient, for early detection of existing risks and for appropriate setting of alarm limits. In this context, both vital parameters of a patient or patient status values, device settings, examination results and also available personnel and technical resources should generally be usable with the system specified during an evaluation. Statements about the current condition of a patient should be made particularly quickly, reliably and as objectively as possible, and it should be possible to adjust the required alarm limits with a high degree of certainty as needed. In this context, the system specified should exploit the potential of networking various medical devices, such as those commonly used in wards to monitor patients, in order to further increase patient safety while reducing the length of stay of a patient in the hospital, on the one hand, and to reduce the burden on treatment staff, on the other.

The task explained above is solved with a system according to the invention and a process according to the invention. Advantageous embodiments of the invention are explained in more detail in the following description with partial reference to figures.

The invention relates to a system for monitoring a patient having at least one evaluation unit which has an interface arrangement comprising at least one first interface which is suitable for receiving patient status values in parallel or serially from at least two medical devices and making them available in the evaluation unit. The evaluation unit is further configured to generate a signal with patient-specific information, taking into account the received patient status values and at least one further setpoint and/or actual value, and to transmit this signal to a receiving unit via the interface arrangement that comprises the first and/or at least one second interface. According to the invention, the system is further configured in such a way that the evaluation unit has a suggestion module which is configured to determine at least one suggested value which corresponds to a limit value for at least one patient status value on the basis of the patient status values obtained from at least two medical devices and the further setpoint and/or actual value, and to transmit this suggested value to the receiving unit via the interface arrangement that comprises the first and/or the at least one second interface.

With the system according to the invention, it is thus possible to carry out an evaluation on the basis of the patient status values generated by at least two medical devices or other data provided by these medical devices and/or other subsystems, whereby a suggested value for a suitable alarm limit value is determined and transmitted to the receiving unit. The suggested value determined according to the invention is preferably provided to a user, for example by means of a receiving unit comprising a display, a loudspeaker, a pager and/or a cell phone. Alternatively, or in addition, it may be provided that the receiving unit is configured in such a way that the suggested value is transmitted to at least one medical device via a unidirectional—or bidirectional data transmission link. In this case, the suggested value is preferably made available to a control unit of the medical device, so that device settings and, in particular, limit values (threshold values) can be changed, for example, on the basis of the suggested value, and an alarm signal is output when these values are reached, exceeded or undershot.

The system according to the invention is thus based on the networking of at least two medical devices which, at least from time to time, record patient status values and transmit them to an evaluation unit which is configured to analyze the patient status values and generate a suggested value for a suitable alarm limit value of at least one patient status value.

In general, it is thus possible to use the information available in the network to change an alarm limit value stored in a medical device connected to the network, either automatically or semi-automatically, for example after renewed confirmation by an operator.

With the system according to the invention, it is thus possible in a preferred manner to acquire and analyze measured values from patients with different medical devices that are located in a common network and to generate suggested values for alarm limits, which are then transmitted to one or more connected medical devices intended for patient monitoring. The respective suggested alarm limits or alarm corridors, which are limited by a minimum and a maximum permissible limit value, or the suggested changes are accepted by an operator at the relevant medical device, for example by pressing a confirmation button. Likewise, it may be provided that a proposed alarm limit or alarm limit change does not have to be confirmed directly at an individual medical device, but rather the confirmation takes place centrally at a monitoring unit of a hospital or intensive care unit provided for this purpose. If necessary, the modified alarm limit can also be accepted automatically, especially if all the prerequisites for the required patient safety are met. This includes, for example, secure data or signal transmission and intermediate validation of the suggested value for an alarm limit.

In a particular embodiment of the invention, the evaluation unit and/or the suggestion module is configured to generate a trend analysis of at least one patient status value based on the patient status values obtained. Furthermore, it is advantageous if the suggestion module is configured to take into account a result of the trend analysis, i.e. a change over time of a relevant patient status value, when determining the suggested value. According to this particular embodiment of the invention, it is possible to take the trend of a patient status value into account when determining a suggested value for an alarm limit, in order to make a suitable change to settings of a medical device, in particular to at least one stored alarm limit, comparatively early even before a limit value or a critical state of health of the patient is reached.

Furthermore, it may be provided that the suggestion module is configured to generate the suggested value taking into account at least one predefined target range or to generate and output a target range for an alarm limit value as a suggested value. A target range is delimited by minimum and maximum permissible limit values of a patient status value. A target range to be considered by the suggestion module when generating a suggested value may further be provided by a data memory of a medical device, from a database, and/or by a patient data management system. A predetermined target range can be used to ensure in a particularly simple manner that the generated suggested value does not fall outside of a permissible range.

A special further development of the invention provides that the medical device comprises a ventilator, a syringe pump, an ECG device, an inhaler, an EMG device, an EIT device, a heart-lung machine, a patient monitor, an imaging examination device and/or a database in which data specific to a patient are stored, workflows in a hospital are stored and/or to at least one of the medical devices are stored. The invention thus enables, in a particular way, the effective evaluation and use of measured values and/or other data provided by medical devices, data storage devices, databases, laboratory devices and/or other subsystems connected to a network. In this case, very different elements can be part of the network and measured values or data generated or provided in other ways can be used to generate at least one suggested value in a suggestion module of an evaluation unit. The generated suggested values are in turn suitable for verifying or changing settings, in particular alarm limits, in at least one of the medical devices connected to the network.

According to a further embodiment, it is provided that the receiver unit is configured as a display of a patient monitor, a ventilator, a syringe pump, a pager, a cell phone, a heart-lung machine and/or a monitor of a central monitoring station. According to this embodiment, the receiving unit is thus a display unit via which information generated in the evaluation unit, in particular a suggested value for an alarm limit value (alarm threshold value) generated by the suggestion module, is visually displayed to the treatment personnel. Furthermore, it is conceivable both to send corresponding information or a suggested value to at least one display unit and to display it at the display unit, and to transmit it to a medical device, so that at least one setting for a control unit of the medical device, in particular an alarm limit, is changed on the basis of the suggested value. Furthermore, it is additionally conceivable that the receiving unit also enables an acoustic output of the suggested value.

In a further embodiment of the invention, at least one data storage unit or database is provided in which the patient status values, setpoint and/or actual values, suggested values and/or other information and values specific to a patient, work processes in a hospital and/or for at least one of the medical devices can be stored at least temporarily and transmitted to the evaluation unit and/or to the suggestion module. This is a data storage unit or database which is arranged centrally, for example in the area of a monitoring station or a computer center and/or decentralized in at least one medical device of a network, and in which at least one value is stored which is used in the evaluation of patient status values, examination results, laboratory values and/or the generation of suggested values. In this context, it is quite conceivable to arrange the data storage unit described above and/or the evaluation unit configured according to the invention at a central location, such as a hospital computer center or a cloud.

According to a further embodiment, it is provided that the suggestion module is configured to generate the suggested value taking into account values stored in at least one look-up table. The look-up table is configured in such a way that input values, in particular unprocessed or at least partially processed patient status values and/or suggested values, are assigned a specifically suitable output value. Such a specific output value may, for example, be the value of a setting of a medical device, in particular an alarm limit value for a patient status value and/or a target range which is limited by a maximum and a minimum permissible limit value.

Furthermore, it is advantageous if a system implemented according to the invention has at least one interface for a data connection to a central hospital ward and/or hospital network. In this case, a particularly effective exchange of data and a reliable generation of suggested values is possible, since the automated generation of suggested values can take place taking into account a particularly large amount of data. In particular, a reliable determination of suggested values can take place when the treatment or examination of a patient is followed with other medical devices and/or the patient is transferred to another bed or another hospital ward. In this way, comprehensive monitoring of a patient, in particular across wards, is ensured.

In addition to the system described above, the invention also relates to a process for generating a suggestion for at least one alarm limit when monitoring a patient who is being treated at least partially with an at least partially automated device. In this process, patient status values are acquired from at least two medical devices and transmitted to an evaluation unit. On the basis of the transmitted patient status values and at least one further setpoint and/or actual value, for example a specific limit value or an indicator for a specific disease, a signal with patient-specific information is generated and transmitted to a receiving unit. According to the invention, the process is characterized in that, on the basis of the patient status values obtained from at least two medical devices and the further setpoint and/or actual value, at least one suggested value corresponding to a limit value for at least one patient status value is determined and transmitted to the receiving unit. The process according to the invention is thus based in turn on the fact that, on the basis of patient status values and desired and/or actual values which are provided from at least two, preferably a plurality, of medical devices of a network, suggested values are generated and transmitted to at least one of the medical devices of the network. The evaluation of the data for generating at least one suggested value can be carried out decentrally in one of the medical devices or a subsystem of the network or centrally in a monitoring station, a central computing unit and/or a central computing center.

Preferably, the suggested value is used to realize a demand-driven control of at least one of the medical devices, in particular for setting or adjusting an alarm limit for at least one patient status value.

Furthermore, a special further development of the process according to the invention provides that, when determining the suggested value, patient-specific data provided by a patient monitor, an ECG device, an EMG device, an EIT device, a ventilator, a syringe pump, a device for imaging examinations, a database, a laboratory database, a hospital monitoring unit, and/or a patient data management system (PDMS), provided patient-specific data, in particular patient status values, device-specific data, and/or data with information about hospital workflows, time of day, and/or personnel resources are taken into account.

This embodiment of the invention is thus based in turn on the data networking of a plurality of medical and other monitoring devices or other subsystems of the network, the patient status values provided in each case as well as target and/or actual values being used to generate at least one suggested value, in particular for an alarm limit, and to transmit this suggested value to a display unit and/or to a controller of one of the medical devices.

A further special further development of the process according to the invention is characterized by the fact that a target range, which is limited by a maximum and a minimum limit value of at least one patient status value, is taken into account when determining the suggested value.

It is also advantageous if the suggested value is determined by the suggestion module of the evaluation unit assigning a value stored in a look-up table to a value determined by calculation and/or to a patient status value. Alternatively, or in addition, it is conceivable to use elements of artificial intelligence at least in part to determine the suggested value. In this context, it is conceivable, for example, that the generation of the suggested value is carried out at least in part with an algorithm that has been taught with the aid of special training data. Preferably, the algorithm is configured in such a way that it also undergoes a learning or optimization process during the generation of suggested values in order to achieve a further improvement in the generation of suggested values.

A further embodiment of the process according to the invention provides that a trend of a patient status value, a condition of at least one of the medical devices, a time of day, information about previous illnesses of the patient, the number and qualification of the medical personnel present and/or a workload of a hospital ward are taken into account when determining the suggested value. Furthermore, a special further development provides that at least temporarily a suggested value is transmitted to the receiving unit of a selected medical device, which suggested value was determined taking into account patient status values transmitted by at least one other medical device.

In the following, without limiting the general idea of the invention, the invention is explained in more detail by means of specific embodiments with reference to the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a system according to the invention for monitoring a patient, and including a network comprised of a plurality of medical devices and subsystems;

FIG. 2 is a schematic view of the structure of a suggestion module; and

FIG. 3 is a view of a look-up table.

### DESCRIPTION OF PREFERRED EMBODIMENTS

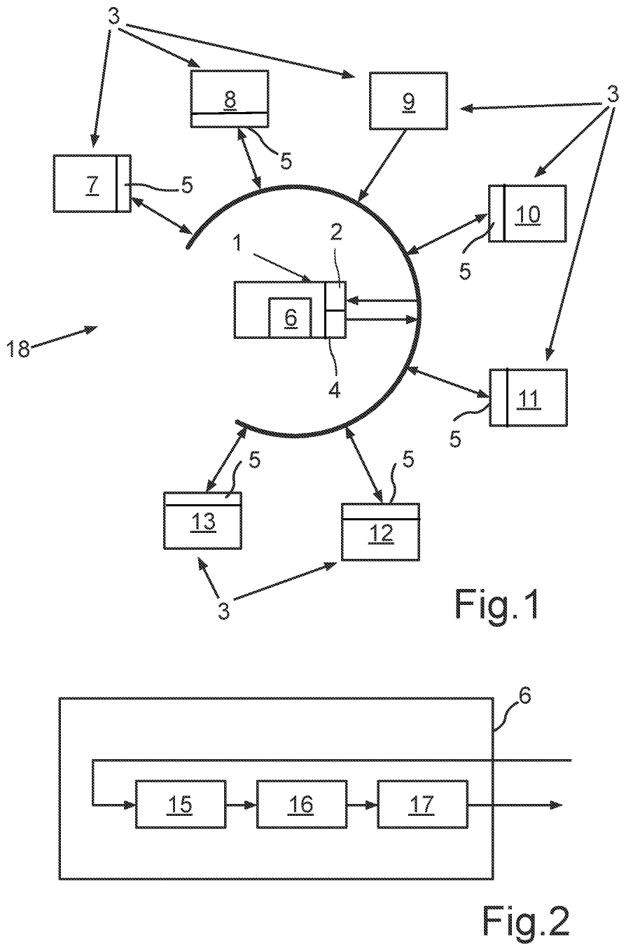

Referring to the drawings, FIG. 1 shows a schematic diagram of a system configured according to the invention for monitoring a patient, which is implemented with a network 18, in which a plurality of network participants, in particular of medical devices 3 and subsystems for data storage, provision, input and/or output, are connected to one another by data technology. The network 18 is configured in such a way that the various network participants can exchange patient-specific and device-specific data as well as other data containing information about work processes and duty schedules of a hospital. Among other things, a variety of patient condition data is available in the network, which is recorded by the various medical devices 3 or otherwise provided, for example, by a patient data management system, by a laboratory database and/or by manual input.

An evaluation unit 1 is provided as a component of the network 18, which can also exchange data, in particular patient status values, setpoint and limit values (threshold values), information about device settings, and suggested values with one or a plurality of network nodes. The data communication takes place unidirectionally—or bidirectionally as required. For this purpose, the evaluation unit has an interface arrangement comprised of at least a first interface for receiving data relevant to the evaluation and a second interface for outputting to the network 18 at least some of the values generated by it, in particular suggested values. Both interfaces 2, 4 of the interface arrangement can be configured separately or as a common interface.

Connected to the network 18 shown in FIG. 1 are each of a patient monitor 10, a central monitoring unit 7, a syringe pump 11, an EIT device 12, a ventilator 13, a patient data management system (PDMS) 8, and a device for blood gas analysis 9 and/or to a data storage unit for the results of blood gas analyses. Of course, depending on the condition of a patient as well as the required treatment and therapy steps, the type and number of medical devices 3 coupled to the network 18, the subsystems as well as the data to be exchanged can be changed as needed.

In the embodiment shown, the device for blood gas analysis 9, the patient monitor 10, the EIT device 12 and the ventilator 13 in particular collect patient status values and make them available to the network 18 or the network participants together with current device settings, in particular alarm limits. The corresponding values can then preferably be displayed via a receiving unit 5 configured as a display unit or a display of the central monitoring unit 7 and/or stored in the patient data management system 8. In other respects, the syringe pump 11 provides data on the current dosage of drugs or active ingredients and/or on a future intended dosage.

All data present in the network 18 can be output on suitable displays of the network participants, and thus both decentrally, via the medical devices 3, and centrally, in the monitoring unit 7, and are thus available to the treatment personnel at different locations. In addition, at least the patient status values provided by the various network participants are made available to an evaluation unit 1. The evaluation unit 1 has a suggestion module 6 which generates at least one suggested value for an alarm limit value based on the patient status values and further data, which in particular contain setpoint and/or actual values, and in turn makes this suggested value available to the network 18 with its network participants. In general, the suggestion module 6 is able to generate at least one suggested value for a limit value taking into account different data, which may include patient status values as well as data containing other patient-specific information, such as gender, age, previous illnesses, as well as device-specific and hospital-specific information, in particular about hospital processes, work schedules, the staffing level in the intensive care unit, and the qualifications of the available treatment personnel.

Taking into account the various data made available to the evaluation unit 1, the suggestion module 6 generates at least one suggested value which corresponds to at least one limit value for a patient status value at which an alarm is to be issued, and transmits this suggested value via the network 18 to a receiving unit 5 of at least one of the network subscribers, in particular to one of the medical devices 3 for which the suggested value is relevant. In this medical device 3, based on the suggested value, a set alarm value, if it deviates from the suggested value, is changed, in particular set to the suggested value, either automatically or after confirmation by an operator. If the suggested value is to be adopted as the new alarm limit value only after confirmation, the suggested value is displayed via a receiver unit 5 configured as a display unit, in particular a display, and adopted in the control of the medical device 3 after confirmation by the treatment personnel.

The evaluation unit 1 is connected to the individual network subscribers via at least one unidirectional- and/or bidirectional interface, which is a first interface 2 for receiving data and a second interface 4 for outputting data. In addition to measured values, in particular patient status values, data acquired by a patient monitor 10 and a ventilator 13, data from the blood gas analysis 9 and the patient data management system 8, but also further information originating from devices for imaging examination of a patient, such as an EIT device 12 or a computer tomograph (CT), and even medication data from syringe pumps 11 can thus be used for determining suitable alarm limits. Furthermore, it is possible to evaluate values entered by clinical treatment staff via a screen terminal of a central monitoring unit 7.

Quality attributes, in this case a Signal Quality Index, are also evaluated to check the plausibility of the data transmitted via the network 18. This prevents incorrect adjustment of alarm limits due to falsified measured values, for example because a sensor worn on the patient's finger for measuring oxygen saturation in the blood (SpO2 sensor) has slipped. The quality attributes are either generated by a sensor of a medical device 3 or calculated on the basis of special algorithms in a signal evaluation unit.

The evaluation unit 1 with the suggestion module 6, represents an intelligent unit, which is optionally implemented on a medical device 3, a data processing unit of a hospital, for example a computer of a hospital ward, or on a central, higher-level data processing unit, which is arranged in a computer center of the hospital or outside the hospital. In particular, the evaluation unit 1 can be integrated into a medical device 3 or implemented in a computer as a PC solution in a network, as shown in FIG. 1. Cloud solutions may also be provided, which offer the advantage of working independently of location and cost-efficiently with high availability.

FIG. 2 is a schematic representation of a suggestion module 6 as provided in accordance with the invention in an evaluation unit 1, which is located together with a plurality of medical devices 3 in a network 18. The suggestion module 6 essentially has an interface arrangement that is comprised of a first interface 2 via which data, in particular patient status values and other patient-specific and device-specific data, are transmitted. In the embodiment example explained herein, physiological and non-physiological measured values and other values, including quality attributes, hospital-specific data, laboratory data, image data, device settings, alarm limits, information about alarms that have occurred, device conditions, and hospital-specific data, such as nursing and shift schedules, are available to the suggestion module 6 for generating a suggested value. The provided data is collected in a data recording unit 15 and structured for further processing.

In a data analysis unit 16, further information provided via the network 18 is added to the data required for an evaluation. This includes, in particular, information about work processes at the respective hospital ward that affect the daily routine and thus the nursing processes, such as visiting, and rest times or time periods scheduled for rounds.

Further information added in the data analysis unit 16 includes day and night information, changes in the patient condition, deviations of the measured values from a target range limited by just permissible minimum and maximum limit values, and trend information relating to the patient status values to be evaluated. Finally, suggested values corresponding to alarm limits of individual patient status values are generated from the analyzed data in a suggestion unit 17 of the suggestion module 6 and made available to the network participants via the network 18. The suggested values are preferably determined via a rule-based system in which, in particular, one or a plurality of look-up tables 14 are stored. Alternatively, or additionally, algorithms utilizing neural networks are used to generate suitable suggested values.

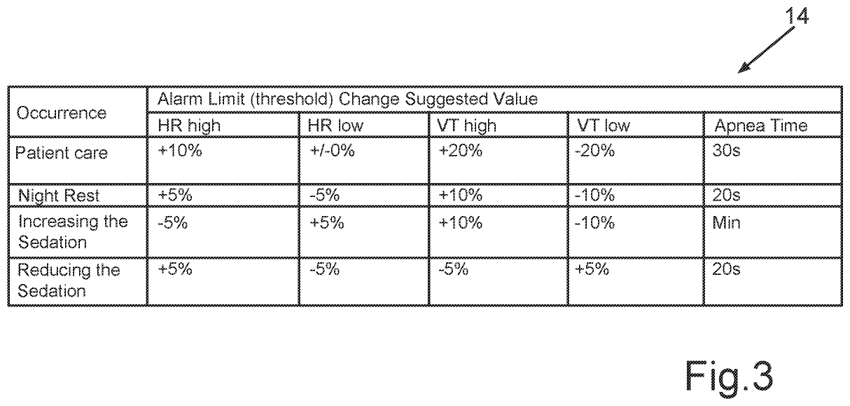

For this purpose, FIG. 3 shows a possible look-up table 14 in which certain events that occur or may occur during the treatment of a patient are assigned suggested values for a change in alarm limits. In FIG. 3, the left column is the detected or input occurrence, such as the noted row headings of the occurrence of patient care, such as a doctor, nurse of other care, the occurrence of night rest, the occurrence of an increase in sedation, and the occurrence of a reduction of sedation. The top row is the alarm limit (the threshold) change suggested value for various suggested alarm limit changes, such as the noted column headings of high heart rate (HR) threshold, low heart rate (HR) threshold, high tidal volume (VT) threshold, and low tidal volume (VT) threshold. The change in assigned suggested values as is noted in FIG. 3. The specified suggested values may be output directly to the network 18 and/or used to generate other suggested values. Additional rules and suggested values for alarm limits may be added to the look-up table 14 for handling multiple simultaneous events.

Moreover, it may be provided that such a look-up table 14 defines both diagnosed disease processes, such as the development of sepsis, and standard operating procedures (SOPs).

The determination of a suggested value corresponding to an alarm limit of a patient status value and the associated initiation of an alarm limit adjustment at at least one medical device 3 of a network 18 may be performed based on the following criteria:
cyclically at certain time intervals, for example every 5 minutes,
when an alarm occurs,
after a trend has settled in and is neither rising nor falling,
after a setting has been changed,
as soon as new information is available from the patient data management system,
after a treatment maneuver has been performed on the patient,
after a medication has been administered or changed and/or
after a nursing procedure, such as repositioning, has taken place on the patient.

The extent to which a suggested value generated by the suggestion module is adopted by a medical device in the network depends on its configuration. As a rule, user interaction is required for the adoption of a suggested value as a new alarm limit value, whereby the treatment staff is first alerted to the arrival of new suggested values for alarm limits by means of an acoustic and/or visual signal. With the aid of the system according to the invention, which uses a large amount of data available in a network, an effective, targeted, but also safe adjustment of alarm limits set in various medical devices is thus achieved.

While specific embodiments of the invention have been shown and described in detail to illustrate the application of the principles of the invention, it will be understood that the invention may be embodied otherwise without departing from such principles.

### LIST OF REFERENCE CHARACTERS

1 Evaluation unit
2 First interface
3 Medical device
4 Second interface
5 Receiver unit
6 Suggestion module
7 Central monitoring unit
8 Patient Data Management System (PDMS)
9 Blood gas analyzer
10 Patient monitor
11 Syringe pump
12 EIT device
13 Ventilator
14 Look-up table
15 Data recording unit
16 Data analysis unit
17 Proposal unit
18 Network

What is claimed is:

1. A system for monitoring a patient, the system comprising:
an interface arrangement comprising at least a first interface, the interface arrangement being configured to receive in parallel or serially patient status values from a plurality of medical devices and to make the received status values available;
a receiving unit;
an evaluation unit configured to receive the status values made available by the interface arrangement and to generate a signal with patient-specific information based on the received patient status values and at least one further setpoint and/or actual value, and to transmit the generated signal to the receiving unit via the interface, the evaluation unit comprising a suggestion module configured to determine at least one suggested value, which corresponds to a limit value for at least one patient status value, based on the patient status values obtained from the plurality of medical devices and the further setpoint and/or actual value, and to transmit the suggested value to the receiving unit via the interface arrangement.

2. A system according to claim 1, wherein the evaluation unit is configured to generate a trend analysis for at least one of the patient status values based on received patient status values, and the suggestion module is configured to determine the suggested value based on the trend analysis.

3. A system according to claim 1, wherein the suggestion module is configured to determine the suggested value based on at least one predetermined target range.

4. A system according to claim 1, wherein each medical device of the plurality of medical devices comprises one or more of a ventilator, a syringe pump, an ECG device, an inhaler, an EMG device, an EIT device, a heart-lung machine, a patient monitor, an imaging examination device and a database in which data specific to a patient, data of work procedures in a hospital and/or data related to at least one of the medical devices are stored.

5. A system according to claim 1, wherein the receiving unit comprises at least one of a display monitor input interface, a display monitor display, a ventilator, a syringe pump, a pager, a cell phone, a heart-lung machine and a monitor of a central monitoring unit.

6. A system according to claim 1, further comprising a data storage unit in which the patient status values, setpoint and/or actual values, default values and/or other values which are specific to a patient, work processes in a hospital and/or to at least one of the medical devices is stored at least temporarily and can be transmitted to the evaluation unit.

7. A system according to claim 1, wherein the suggestion module is configured to generate the suggested value based on values stored in at least one look-up table.

8. A system according to claim 1, further comprising the plurality of medical devices.

9. A system according to claim 1, further comprising a data connection interface configured to data connect to a central hospital ward and/or hospital monitoring system.

10. A process for generating a proposal for at least one alarm limit in monitoring of a patient who is at least partially treated with an at least partially automated medical device, the process comprising the steps of:
detecting patient status values by a plurality of medical devices;
transmitting the detected patient status values to an evaluation unit;
generating a signal with patient-specific information based on the transmitted patient status values and at least one further setpoint and/or actual value;
transmitting the signal to a receiving unit;
determining at least one suggested value which corresponds to a limit value for at least one patient status value based on the patient status values obtained from the plurality of medical devices and the further setpoint and/or actual value; and
transmitting the at least one suggested value to the receiving unit.

11. A process of claim 10, wherein the determined suggested value is based on patient status values and/or other data transmitted from a patient monitor, an ECG device, an EMG device, an EIT device, a ventilator, a syringe pump, a device for imaging examination of a patient, a patient database, a database with data on duty schedules and the workflows of a hospital or a nursing facility, a laboratory database and/or from a hospital control room.

12. A process according to claim 10, wherein the determined suggested value is based on a target range for the limit value corresponding to at least one patient status value.

13. A process according to claim 10, wherein the suggested value is determined by a look-up table assignment of a suggested value to be transmitted to the receiving unit to an at least partially computationally determined value.

14. A process according to claim 10, wherein the determined suggested value is based on a trend of a patient status value, a condition of at least one of the medical devices, a time, a patient condition, a number and qualification of the medical personnel present and/or a number and condition of further patients.

15. A process according to claim 10, wherein at least temporarily a suggested value is transmitted to the receiving unit of a selected medical device, which suggested value is determined based on patient status values transmitted by at least one other medical device.
